(19) United States
(12) Patent Application Publication
(10) Pub. No.: US 2005/0142095 A1
(43) Pub. Date: Jun. 30, 2005

(54) Cosmetic compositions containing meadowsweet extract and methods for treating skin

(75) Inventors: Neil D. Scancarella, Wyckoff, NJ (US); Gale McElroy Reinhart, Middletown, NJ (US); Julio Gans Russ, Westfield, NJ (US)

Correspondence Address: Julie Blackburn, Esq.;Revlon Consumer Products Corporation, Law Dept., 237 Park Avenue, New York, NY, 10017, US

(73) Assignee: Revlon Consumer Products Corporation, New York, NY (US)

(21) Appl. No.: 11/070,631

(22) Filed: Mar. 3, 2005

Related U.S. Application Data
(62) Division of application No. 10/117,632, filed on Apr. 5, 2002.

(51) Int. Cl. A61K 7/06; A61K 35/78

(52) U.S. Cl. 424/74; 424/725

(57) ABSTRACT

A cosmetic composition for ameliorating the adverse effects of aging on skin or hair comprising a cosmetically effective amount of meadowsweet extract in a cosmetically acceptable carrier; a method for ameliorating the adverse effects of aging and a method for exfoliating skin with cosmetic compositions containing an effective amount of meadowsweet extract.

### TECHNICAL FIELD

The invention is in the field of cosmetic compositions for treating skin and hair for improvement, and to ameliorate the adverse effects of aging.

### BACKGROUND OF THE INVENTION

Certain properties such as skin laxity, blotchiness, yellowing, and defects in keratinization are often seen in aging skin. It is well known that as one ages, cell turnover in skin significantly decreases. This means that superficial keratinous cells remain on the skin surface longer, which in turn contributes to the yellowish texture often seen in aged skin. It is well known that exfoliating the skin will improve cell turnover as it is the body's natural reaction to synthesize new cells to replace those that are removed in the exfoliation process. However, in order to get optimum exfoliation it would be necessary to cleanse the skin with physical exfoliants on the skin several times per day. Since this is not practical, it makes more sense to incorporate ingredients with exfoliating properties directly into cosmetics such as skin creams, lotions, and foundation makeup that are applied to skin and remain for hours at a time before being removed. In this way, the exfoliant remains on the skin for extended periods of time and can interact with the superficial keratinous cells to cause exfoliation.

It is also known that aging causes certain undesirable changes in hair. Not only does hair gray, but it becomes more prone to breakage, stiffness, and loss of body.

With respect to treatment of skin, many of the known exfoliating agents, particularly alpha hydroxy acids, are medium strength acids. Not only do they sometimes cause skin irritation and sensitivity, these ingredients may be difficult to formulate with because they may be reactive with many of the ingredients traditionally used in cosmetic compositions, especially pigments. Accordingly, there is a need for cosmetic compositions containing an exfoliating ingredient that is capable of ameliorating the adverse effects of aging. This ingredient will ideally provide the benefits associated with stronger chemical exfoliants, but without the irritation. In addition, the ingredient will be easy to formulate into cosmetic products.

It is an object of the invention to provide a skin care composition for ameliorating the adverse effects of aging comprising applying a cosmetic composition containing meadowsweet extract (spiraea ulmaria) to the skin.

It is a further object of the invention to provide a method for exfoliating skin comprising applying to the skin a composition containing meadowsweet extract in a cosmetically acceptable carrier.

It is a further object of the invention to provide a skin care composition containing meadowsweet extract.

### SUMMARY OF THE INVENTION

The invention comprises a cosmetic composition for ameliorating the adverse effects of aging comprising a cosmetically effective amount of meadowsweet extract in a cosmetically acceptable carrier.

The invention further comprises a method for exfoliating skin comprising applying to the skin an effective amount of an extract of meadowsweet extract in a cosmetically acceptable carrier.

The invention further comprises a method for ameliorating the adverse effects of aging on the skin by treating the skin with a cosmetic composition containing an effective amount of an extract of meadowsweet.

### DETAILED DESCRIPTION

The spiraea ulmaria or meadowsweet extract that is used in the claimed compositions and method is an extract obtained from the spiraea ulmaria plant, which is a perennial herb with a tough, erect, branched, leafy stem and white flowers. Meadowsweet is found in damp woods and meadows throughout Europe. The extract may be obtained from the leaves, flowers, seeds, branches, or stems, but is most preferably obtained from the flowers. The extract may be prepared according to well known methods for preparing botanical extracts. In addition, meadowsweet extract is commercially available from a variety of sources such as Active Concepts, South Plainfield, N.J. Generally, the amount of meadowsweet extract required to achieve the desired benefits of the composition ranges from about 0.0001 to 35%, preferably about 0.005 to 20%, more preferably about 0.01-15% by weight of the total cosmetic composition.

The extract may be included in a wide variety of cosmetic compositions, including creams, lotions, sunscreens, color cosmetic products such as mascaras, blushes, eyeshadows; hair care products such as shampoos, hair conditioners, and the like. The cosmetics may be anhydrous, or in the form of a water and oil emulsion or solution form.

Lotions, Creams, Gels, and Sunscreens

The meadowsweet extract may be used to make skin care products such as lotions, creams, gels, and sunscreens.

Suitable skin care lotions and creams are in the emulsion form, and may be water-in-oil or oil-in-water emulsions, preferably oil-in-water emulsions. Creams, lotions, and/or may contain the following ranges of ingredients:
about 0.01-50% meadowsweet extract,
about 0.5-95% water,
about 0.1-90% oil, and
about 0.01-20% surfactant.

### Oils

If present, suggested ranges for such oils are about 0.1-90%, preferably 0.5-40%, more preferably 1-35% by weight of the total composition. The oils used may be volatile or nonvolatile. The term “volatile” means that the oil has a measureable vapor pressure, or a vapor pressure of at least 2 mm. of mercury at 20° C. The term “nonvolatile” means that the oil has a vapor pressure of less than 2 mm. of mercury at 20° C. Suitable volatile solvents generally have a viscosity of 0.5 to 10 centistokes at 25° C. Suitable volatile oils include linear silicones, cyclic silicones, paraffinic hydrocarbons, or mixtures thereof. Cyclic silicones (or cyclomethicones) are of the general formula: 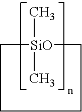
where n=3-6.

Linear volatile silicones in accordance with the invention have the general formula:
(CH₃)₃Si—O—[Si(CH₃)₂—O]ₙ—Si(CH₃)₃
where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

Also suitable as the volatile oils are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic ydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Transfer resistant cosmetic sticks of the invention will generally comprise a mixture of volatile silicones and volatile paraffinic hydrocarbons.

A wide variety of nonvolatile oils are also suitable for use in the cosmetic compositions of the invention. The nonvolatile oils generally have a viscosity of greater than 10 centipoise at 25° C., and may range in viscosity up to 1,000,000 centipoise at 25° C. Examples of nonvolatile oils suitable for use in the cosmetic sticks of the invention include esters of the formula RCO—OR′ wherein R and R′ are each independently a C₁₋₂₅, preferably a C₄₋₂₀ straight or branched chain alkyl, alkenyl or alkoxycarbonylalkyl or alkylcarbonyloxyalkyl. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, and fatty alcohols such as oleyl alcohol, isocetyl alcohol, and the like, as well as the esters disclosed on pages 24-26 of the C.T.F.A. Cosmetic Ingredient Handbook, First Edition, 1988, which is hereby incorporated by reference.

The oil may also comprise naturally occuring glyceryl esters of fatty acids, or triglycerides. Both vegetable and animal sources may be used. Examples of such oils include castor oil, lanolin oil, triisocetyl citrate, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable as the oil are synthetic or semi-synthetic glyceryl esters, e.g. fatty acid mono-, di-, and triglycerides which are natural fats or oils that have been modified, for example, acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, and so on.

Also suitable as the oil are nonvolatile hydrocarbons such as isoparaffins, hydrogenated polyisobutene, mineral oil, squalene, petrolatum, and so on.

Straight or branched chain fatty alcohols having the formula R-OH, wherein R is a straight or branched chain saturated or unsaturated alkyl having 6-30 carbon atoms, are also suitable oils. Such fatty alcohols include cetyl alcohol, stearyl alcohol, cetearyl alcohol, and the like.

Also suitable as the oil are various lanolin derivatives such as acetylated lanolin, acetylated lanolin alcohol, and so on.

Nonvolatile silicones, both water soluble and water insoluble, are also suitable as the oil component. Such silicones preferably have a viscosity of 10 to 600,000 centistokes, preferably 20 to 100,000 centistokes at 25° C. Suitable water insoluble silicones include amodimethicone, bisphenylhexamethicone, dimethicone, hexadecyl methicone, methicone, phenyl trimethicone, simethicone, dimethylhydrogensiloxane, stearoxytrimethylsilane, vinyldimethicone, and mixtures thereof. Also suitable are water soluble silicones such as dimethicone copolyol, dimethiconol, and the like. Such silicones are available from Dow Corning as the 3225C formulation aid, Dow 190 and 193 fluids, or similar products marketed by Goldschmidt under the ABIL tradename.

Also suitable as the nonvolatile oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin, are also suitable shine enhancers.

Guerbet esters are also suitable oils. The term “guerbet ester” means an ester which is formed by the reaction of a guerbet alcohol having the general formula: 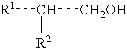
with a carboxylic acid having the general formula:
R³COOH, or
HOOC—R³—COOH
wherein R¹ and R² are each independently a C₄₋₂₀ alkyl and R³ is a substituted or unsubstituted fatty radical such as a C₁₋₅₀ straight or branched chain saturated or unsaturated alkyl or alkylene, or phenyl, wherein the substituents are halogen, hydroxyl, carboxyl, and alkylcarbonylhydroxy. Particularly preferred is a carboxylic acid wherein the R group is such to provide an ingredient known as meadowfoam seed oil.

Preferably, the guerbet ester is a fluoro-guerbet ester which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. Most preferred is a guerbet ester having the tentative CTFA name fluoro-octyldodecyl meadowfoamate. This ester is sold by Siltech, Norcross Ga. as Developmental Ester L61125A, under the tradename Silube GME-F.

Preferably, the compositions of the invention are emulsion lotion or cream compositions and contain volatile silicone and nonvolatile silicone, preferably cyclomethicone and dimethicone, phenyl trimethicone, dimethicone, copolymer, or mixtures thereof.

### Surfactant

The compositions of the invention preferably comprise about 0.01-20%, preferably about 0.1-15%, more preferably about 0.5-10% by weight of the total composition of a surfactant. The surfactant is preferably nonionic, although if the composition is in the form of a shampoo or conditioner it will preferably contain anionic or cationic surfactants, respectively. Suitable nonionic surfactants or emulsifiers include alkoxylated alcohols, or ethers, formed by the reaction of an with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products have the general formula: 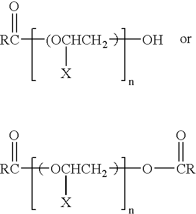
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 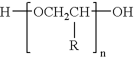
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers which have at least one hydrophilic radical and at least one lipophilic radical. The silicone surfactant used in the compositions of the invention are organosiloxane polymers that may be a liquid or solid at room temperature. The organosiloxane surfactant is generally a water-in-oil or oil-in-water type surfactant which is, and has an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The term “organosiloxane polymer” means a polymer containing a polymeric backbone including repeating siloxy units that may have cylic, linear or branched repeating units, e.g. di(lower)alkylsiloxy units, preferably dimethylsiloxy units. The hydrophilic portion of the organosiloxane is generally achieved by substitution onto the polymeric backbone of a radical that confers hydrophilic properties to a portion of the molecule. The hydrophilic radical may be substituted on a terminus of the polymeric organosiloxane, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the organosiloxane polymer backbone, confers lipophilic properties to the substituted portion of the polymer. Examples of organic radicals which will conver lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interruped by one or more oxygen atoms, a benzene ring, amides, esters, or other functional groups.

The polymeric organosiloxane surfactant used in the invention may have any of the following general formulas:
MₓQ_{y}, or
MₓT_{y}, or
MDₓD′_{y}D″_{z}M
wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RR′SiO_{1.5} or RRSiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:
   MDₓD′_{y}D″_{z}M
   wherein
M=RRRSiO_{1/2}
D and D′=RR′SiO_{2/2}
D″=RRSiO_{2/2}
x, y, and z are each independently 0-1000,
where R is methyl or hydrogen, and R′ is a hydrophilic radical or a lipophilic radical, with the proviso that the compound contains at least one hydrophilic radical and at least one lipophilic radical.
   Most preferred is wherein
M=trimethylsiloxy
D=Si[(CH₃)][(CH₂)ₙCH₃]O_{2/2} where n=0-40,
D′=Si[(CH₃)][(CH₂)ₒ-O-PE)]O_{2/2} where PE is (—C₂H₄O)ₐ(—C₃H₆O)_{b}H, o=0-40, a=1-100 and b=1-100, and
D″=Si(CH₃)₂O_{2/2}

More specifically, suitable silicone surfactants have the formula: 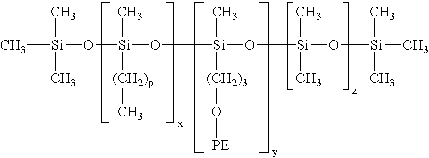
wherein
p is 0-40, and
PE is (—C₂H₄₀)ₐ(—C₃H₆O)_{b}—H
   where x, y, z, a, and b are such that the maximum molecular weight of the polymer is approximately about 50,000.

Another type of preferred organosiloxane emulsifier suitable for use in the compositions of the invention are emulsifiers sold by Union Carbide under the Silwet™ trademark. These emulsifiers are represented by the following generic formulas:
(Me₃Si)_{y-2}[(OSiMe₂)_{x/y}O-PE]_{y}
wherein
PE=-(EO)ₘ(PO)ₙR
R=lower alkyl or hydrogen
Me=methyl
EO is polyethyleneoxy
PO is polypropyleneoxy
m and n are each independently 1-5000
x and y are each independently 0-5000, and 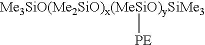
   wherein
PE=—CH₂CH₂CH₂O(EO)ₘ(PO)ₙZ
Z=lower alkyl or hydrogen, and
Me, m, n, x, y, EO and PO are as described above,
   with the proviso that the molecule contains a lipophilic portion and a hydrophilic portion. Again, the lipophilic portion can be supplied by a sufficient number of methyl groups on the polymer.

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

In the preferred compositions of the invention, the nonionic surfactant comprises a silicone surfactant, specifically dimethicone copolyol, either alone or in combination with an alkoxylated alcohol, or ether, formed by the reaction of a fatty acid with a polyhydric alcohol such as glucose or methyl glucose and an ethoxylated alcohol; a sorbitan derivative, and mixtures thereof.

Suitable cationic, anionic, zwitterionic, and amphoteric surfactants are disclosed in U.S. Pat. No. 5,534,265, which is hereby incorporated by reference in its entirety.

### Sunscreens

The compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorpated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)] amnobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate O, (Octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and so on.

### Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### Humectants

Preferably the compositions of the invention comprise 0.01-30%, preferably 0.5-25%, more preferably 1-20% by weight of the total composition of one or more humectants. Suitable humectants include materials glycols, sugars, and similar materials. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

### Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

### Gellants

It may be desireable to include other gellants in the oil or water phase of the composition to provide gelling. Such gellants may be included a range of about 0.1-20%, preferably about 1-18%, more preferably about 2-10% by weight of the total composition is suggested. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

### Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

### Emulsion Stabilizers

It may be desirable to incorporate one or more emulsion stabilizers in the composition. If so, suggested ranges are about 0.0001-5%, preferably about 0.0005-3%, more preferably about 0.001-2% by weight of the total composition. Suitable emulsion stabilizers include salts of alkali or alkaline earth metal chlorides or hydroxides, such as sodium chloride, potassium chloride, and the like.

Skin and Hair Cleansing and Conditioning Compositions

The PPC may be incorporated into skin and hair cleansing and conditioning compositions such as facial cleansers, shampoos, hair conditioners and the like.

Generally skin and hair cleansing compositions comprise:
1-95% water, and
0.1-40% surfactant, preferably an anionic, amphoteric, or zwitterionic surfactant.

Particularly preferred is a facial cleanser comprising:
0.05-10% meadowsweet extract,
0.01-40% surfactant,
   and the remainder water. Preferably the surfactant is a nonionic or anionic surfactant, or mixtures thereof, as mentioned herein.

Suitable hair conditioner compositions comprise:

0.0001-20% meadowsweet extract,
0.1-20% cationic surfactant,
0.1-30% fatty alcohol,
0.001-10% nonionic surfactant, and
5-95% water.

Suitable cationic and nonionic surfactants are as mentioned herein. Examples of suitable fatty alcohols include those having the general formula R—OH, wherein R is a C₆₋₃₀ straight or branched chain, saturated or unsaturated alkyl.

Foundation Makeup, Color Cosmetics

The meadowsweet extract may be used in foundation makeup or color cosmetics such as eyeshadow, blush, concealer, or eyeliner compositions in the liquid, cream, solid, or stick form. Suitable foundation makeup compositions may be water-in-oil or oil-in-water emulsions. Such compositions generally comprise:
0.01-50% meadowsweet extract,
0.5-95% water,
0.5-25% particulate matter,
0.01-20% surfactant, and
0.1-95% oil.

In addition, these composition may further contain ingredients selected from the group of humectants, preservatives, nonvolatile or volatile oils, gellants, and mixtures thereof.

Various anhydrous color cosmetic products may also be suitable, such as blush, powder, lipsticks, eyeshadows, and the like. Such anhydrous color cosmetic compositions may generally comprise:
about 0.0001-20% meadowsweet extract,
about 0.1-99% oil,
about 0.1-85% wax,
about 0.1-80% particulate matter.

### Waxes

Suitable waxes are animal, vegetable, mineral, and synthetic waxes, or silicone waxes including stearoxydimethicone, stearyl dimethicone, polyethylene, paraffin, ceresin, ozokerite, including but not limited to those set forth in U.S. Pat. No. 5,725,845 which is hereby incorporated by reference in its entirety. Preferred ranges of wax are about 0.5-75%, preferably about 1-65% by weight of the total composition.

### Particulate Matter

The makeup or color cosmetic compositions preferably comprises 1-20%, more preferably 1.5-18% of particulate matter having a particle size of 0.01 to 200, preferably 0.25-100 microns. The particulate matter may be colored or non-colored (for example white) non-pigmentitious powders that may give the cosmetic stick an opaque or semi-opaque quality and contribute to stick structure. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetic sticks it is used more for its ability to mute color, and/or provide an opaque or semi-opaque finish, then as a colorizing ingredient. The above mentioned powders may be surface treated with lecithin, amino acids, mineral oil, silicone, or various other agents either alone or in combination, which coat the powder surface and render the particles more lipophilic in nature.

The particulate matter component also may comprise various organic and/or inorganic pigments, alone or in admixture with one or more non-pigmentatious powders. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated.

The invention will be further described in connection with the following examples which are set forth for the purposes of illustration only.

### EXAMPLE 1

A skin care lotion was prepared as follows:

| | w/w % |
|---|---|
| Cyclomethicone, dimethicone, phenyl trimethicone | 3.00 |
| Cyclomethicone, dimethicone copolyol | 12.00 |
| Tocopherol | 0.10 |
| Camilla Sinesis extract | 0.60 |
| Hydroxylated milk glycerides | 1.00 |
| Petrolatum | 5.00 |
| Phenoxyethanol | 1.00 |
| Propylparaben | 0.10 |
| Ethylparaben | 0.10 |
| HDI/trimethylol hexyllactone crosspolymer, silica | 5.00 |
| Silica dimethyl silylate | 1.00 |
| Polyethylene | 3.00 |
| Sodium chloride | 1.00 |
| Butylene glycol | 10.00 |
| Methyl paraben | 0.30 |
| Glycerin | 10.00 |
| Saxifraga sarmentosa extract | 0.50 |
| Water, glycerin, propylene glycol, bacillus ferment | 0.20 |
| Water, Spiraea ulmaris extract | 5.00 |
| Buttermilk powder | 0.10 |
| Lactobacillus milk/calcium/phosphorus/magnesium | 0.05 |
| zinc ferment | |
| Butylene glycol | 0.12 |
| Casein | 0.075 |
| Carrageenan | 0.006 |
| Triethanolamine | 0.009 |
| Water | QS |

The composition was prepared by combining the oil phase ingredients separately. The water phase ingredients were combined. The phases were combined and mixed well.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1-20. (canceled)

21. A method for ameliorating the adverse effects of aging comprising applying to the skin a cosmetic composition containing an effective amount of meadowsweet extract.

22. The method of claim 21 wherein the adverse effects of aging that are ameliorated are blotchy skin, skin laxity, blemishes, and skin yellowing.

23. The method of claim 21 wherein the cosmetic composition is an emulsion.

24. The method of claim 23 wherein the oil phase comprises silicone oil.

25. The method of claim 24 wherein the silicone oil is a volatile linear or cyclic silicone, a non-volatile silicone, or mixtures thereof.

26. The method of claim 25 wherein the composition additionally comprises a volatile paraffinic hydrocarbon.

27. The method of claim 21 wherein the cosmetic composition is in the form of a water and oil emulsion foundation makeup composition.

28. The method of claim 21 wherein the composition additionally comprises one or more humectants.

29. The method of claim 28 wherein the humectants are mono-, di-, or polyhydric alcohols.

30. The method of claim 29 wherein the humectants are present ranging from about 0.1-10% by weight of the total composition.

31. The method of claim 21 wherein the composition comprises one or more gelling agents.

32. The method of claim 31 wherein the gelling agent comprises a wax.

33. The method of claim 31 wherein the gelling agents are present ranging from about 0.01-25% by weight of the total composition.

34. The method of claim 21 wherein the composition further comprises one or more botanical extracts.

35. The method of claim 34 wherein the botanical extracts are present ranging from about 0.001-20% by weight of the total composition.

36. A method for exfoliating skin comprising applying to the skin an effective amount of meadowsweet extract in a cosmetically acceptable carrier.

37. The method of claim 36 wherein the meadowsweet extract is from the flower.

38. The method of claim 36 wherein the composition is a water and oil emulsion.

39. The method of claim 38 wherein the oil comprises a volatile linear or cyclic silicone, a non-volatile silicone, or mixtures thereof.

40. The method of claim 36 wherein the composition further comprises one or more humectants.
